(19) United States
(12) Patent Application Publication
(10) Pub. No.: US 2014/0067003 A1
(43) Pub. Date: Mar. 6, 2014

(54) SYSTEM AND METHOD FOR AUTONOMIC BLOOD PRESSURE REGULATION

(71) Applicants: Abhi Vase, Los Altos Hills, CA (US); Stephen McMahon, Sydney (AU)

(72) Inventors: Abhi Vase, Los Altos Hills, CA (US); Stephen McMahon, Sydney (AU)

(21) Appl. No.: 13/956,344

(22) Filed: Jul. 31, 2013

Related U.S. Application Data
(60) Provisional application No. 61/678,574, filed on Aug. 1, 2012.
(60) Provisional application No. 61/678,071, filed on Jul. 31, 2012.

(51) Int. Cl. A61N 1/36; A61N 1/05

(52) U.S. Cl. CPC A61N 1/36117; A61N 1/0551
USPC 607/44; 607/118

(57) ABSTRACT

A system for regulating blood pressure by stimulating an afferent pathway to the brain which produces an efferent output in kidneys includes a electrode device adapted for implantation in the cervical region, a stimulator generator, a cable connecting the electrode device and the stimulator generator, wherein the cervical region is generally located between a pair of common carotid arteries, above an aortic arch and in front of cervical vertebrae C2 and C3. A method of implantation includes placing the electrode device in the cervical region, selectively energizing the device in accordance with a stimulation scheme, assessing any changes in the patient's blood pressure, selectively energizing the device in accordance with another stimulation scheme, and assessing any changes in the patient's blood, for determining an optical stimulation scheme, wherein stimulation scheme involves parameters including, for example, position, placement and configuration of the electrode device in relation to surrounding tissue and/or organs, selection of electrodes energized, width, frequency and amplitude of stimulation current.

### CROSS REFERENCE TO RELATED APPLICATIONS

This application claims priority to and the benefit of U.S. Application Ser. No. 61/678,071, filed Jul. 31, 2012, and U.S. Application Ser. No. 61/678,574, filed Aug. 1, 2012, the entire contents of both of which are hereby incorporated by reference.

### FIELD OF INVENTION

The present invention relates generally to implantable medical apparatus, systems and methods for neurovascular stimulation and regulation of blood pressure and/or other physiologic conditions.

### BACKGROUND OF INVENTION

The Problem

Over 150 million Indians were suffering from high blood pressure at the end of 2008-14% of the global burden of uncontrolled hypertension. The prevalence of hypertension varies from about 28-48% in urban areas to as high as 54% in some rural areas in North and South India. Many of these people are undiagnosed and others who have been diagnosed struggle to incorporate diet and exercise into their daily regimen and are non-compliant, which contributes to a worsening disease state.

In the US, the situation is not much better. Approximately a third of the U.S. population had hypertension in 2008 and about 20 million people struggled with uncontrolled resistant hypertension. Hypertension prevalence has also been increasing in other countries, and an estimated 972 million people in the world are suffering from this problem. Incidence rates of hypertension range between 3% and 18%, depending on the age, gender, ethnicity, and body size of the population studied. Despite advances in hypertension treatment, control rates continue to be sub-optimal. Only about one third of all hypertensives are controlled in the United States.

Resistant (or Refractory) HTN is defined as the failure to reach goal BP despite appropriate treatment with full doses of at least 3 anti-hypertensive medications, including a diuretic. This is a significant patient population in whom a device-based therapy could provide a substantial benefit.

Market

Hypertension, defined as systolic/diastolic blood pressure ≧140/90 mm Hg is the number one risk factor for premature death worldwide, affecting roughly one in three adults (˜175M in the United States and developed Europe) and driving a related global annual healthcare expenditure estimated at $500 billion. While hypertension in and of itself can be symptomless it increases the risk of negative outcomes including heart attack, stroke, and death. It is estimated that every 20/10 mm Hg increase in systolic/diastolic blood pressure corresponds to a two-fold increase cardiovascular mortality risk.

Drug therapy for treating hypertension is effective for the majority of patients who comply with medication requirements; however, ˜2% of patients with resistant disease treated for hypertension are on ≧5 drugs, which creates a significant compliance burden, not to mention the risk of side effects and drug-drug interactions (especially in patients with comorbidities). Clinical literature indicates that ˜66% of hypertension patients are treated (˜116M in the United States and developed Europe) which implies that ˜2.7M (2.3%*116M) patients in the United States and developed Europe take five or more medications to treat hypertension. Conservative estimates of this population in India, peg this number somewhere between 5-8M. The initial target population for our therapy is this group of patients.

New Innovations in Hypertension’

Recently, new and promising neuromodulation-based medical device therapies have provided amazing efficacy in a number of short trials (˜3-5 years). While additional research is necessary prior to unequivocal endorsement of the therapy, the results obtained over the past decade are striking. With reference to FIG. 1, these therapies rely on autonomic neuromodulation—stimulation of the parasympathetic nervous system or blocking the sympathetic nervous system.

With reference to FIG. 2, an existing system and device is adapted to perform renal sympathetic denervation. This therapy is priced at approximately $5000 for the devices. With reference to FIG. 3, another system and device rely on stimulation of the carotid sinus (baroreflex) and is expected to be priced at $15,000-20,000 for the device and electrode. A summary of results obtained with the foregoing known systems as shown in FIG. 4, highlights the efficacy of the therapy over a three year period. While the results are undoubtedly encouraging, both of these approaches are not without their disadvantages.

With renal denervation, disadvantages include:

1. The renal denervation procedure is not reversible

2. Physicians are not aware of their success rate immediately after the ablation is performed. They typically find out whether they successfully ablated the network of renal sympathetic nerves only a month or so after the procedure.

3. The procedure relies on placing an RF catheter inside the 2-4 mm renal artery and then using heat to destroy a network of nerves running around and in the adventitia of the artery. It is unlikely that this procedure does not increase the risk of causing renal stenoses.

With baroreceptor stimulation, disadvantages include:

1. The current device relies on a traditional “lead and can” system that resembles a pacemaker and a lead.

2. Stimulation of the baroreceptors does not consistently result in regulation of blood pressure. Undesirable and unintended side effects are also possible.

3. This system is bulky and expensive.

As these procedures become to be practiced routinely, the time taken to perform the procedure and the potential to cause damage to the endothelial wall of the vasculature may continue to be significant risks to the mass uptake of these therapies. In addition, because the therapy is not reversible and/or is priced out of the range of the average consumer, it will limit the number of people who can benefit from this therapy.

There is a desire for an implantable device to automatically regulate blood pressure that can compete in the broad anti-hypertension therapeutic market, a market that is valued at over $70 B in the US alone. There is also a desire for an implantable device that is much smaller and significantly less expensive, and would enable innovative power management as well as closed-loop blood pressure control.

### SUMMARY OF THE INVENTION

The present invention provides a minimally invasive therapy that is a better alternative to complete neuroablation or renal denervation. The present invention advantageously recognizes the existence of an afferent circuit to the brain located in the cervical region which when stimulated produces an efferent output in at least the kidneys, if not also in other organs in the abdominal region. The present invention also allows for closed-loop blood pressure control which would enable it to compete with anti-hypertensive medication across the board, opening up larger markets because of the significant value proposition of improved compliance with a minimally invasive 30-minute outpatient procedure.

The device targets patients suffering from chronic uncontrolled hypertension as a result of sympatho-adrenal overactivity. It is a reversible focal therapy that enables targeted treatment of a neural circuit that drives hypertension and systemic inflammation in the body. Patients will not have to remember to take meds, and the device will enable seamless control of hypertension as well as reduce overall cardiovascular risk. This technology also has the potential to improve insulin sensitivity and combat worsening diabetes.

Desired outcome(s) include: 1) restoration of sympatho-vagal balance and instantaneous control of blood pressure; 2) reduction in nocturnal blood pressure to improve overall cardiovascular risk; and 3) autoregulation of blood pressure. The device may also stimulate chemosensing ability in the body to help regulate glucose and insulin production.

Advantages over existing devices and systems include: 1) broad application beyond chronic intractable hypertensives; 2) reversibility—nerves are still intact—titrated therapy can serve a broader market—not just chronic uncontrolled resistant hypertensives; 3) reduced risk and incidence of renal artery damage or renal stenoses; 4) faster implantation procedure depending on location and embodiment; and (5) true alternative to drug therapy—addresses compliance.

There are a number of technology innovations that are integrated in the present invention, ranging from flexible electronics and flexible power management to MEMS technology for wireless communication and power management.

For therapy delivery, features and considerations include the Circadian Rhythm, closed-loop control, dose titration to MAP, dose titration to Biomarkers (for example, ANG II, rennin). Various location features and considerations include BP sensor location, implantable stimulation device location, minimally invasive access, injectable percutaneous delivery and nerve targets.

The present invention in one embodiment includes a system for autonomic blood regulation comprising a wireless BP sensor, MEMS BP sensor and MEMS antenna. The present invention may further include power-management circuits for telemetry (between sensor and device, or between device and controller where controller may include iPAD, mobile device or laptop), microstimulator—ultrathin flexible electronics (“electrode and device”), virtual cathode-anode enabled by specific pulse amplitudes and electrode spacing, multiple electrodes, sensor network. The present invention may also include transcutaneous hybrid therapy using virtual electrodes, carrier wave superimposition to activate target from farther away, endocardial or Transvenous delivery tools, MRI compatible device, markers for easy visualization via X-ray, ultrasound or MR, and physician monitoring and compliance software to track adherence

Methods of the present invention include features and considerations including frequencies—blocking and creation of virtual electrodes, amplitude to activate preferential afferents rather than pain fibers or other neural pathways, and stimulation “dosing” schemes. Methods may also include ramping, burst stimulation, anti-tachyphylaxis algorithms.

An implantable neurostimulator device of the present invention may be implanted at any suitable location in a patient's body, including, for example, the cervical region, preferably, near the cervical ganglia, between the common carotid arteries, above the aortic arch, and more preferably, in front of cervical vertebrae C2 and C3, near the carotid junction or carotid sinus. Real time, functional testing methods are implemented during implantation procedure to identify an optimal implantation site in relation to surrounding affected tissues and/or organs, including plexi, such as the Hering's nerve, Vagus nerve, and carotid baroreceptors, and/or the brain stem.

A method of the present invention includes functional testing to identify an ideal implantation site. The function testing includes (1) placing an electrode device with multi-point/multi-channel capabilities in a selected position in the cervical region, (2) applying a first selected stimulation scheme with selected parameters, (3) assessing in real time changes in activity/ies of the kidneys and/or blood pressure of the patient in response to the first selected simulation scheme with selected parameters, (4) applying a second selected stimulation scheme (with different selected parameters), (5) assessing the real time changes in activity/ies of the kidneys and/or blood pressure of the patient in response to the second selected stimulation scheme, and (6) comparing results of the steps (3) and (5) to identify a desired result and stimulation scheme. Through a methodical application of different stimulation schemes, including different permutations of, for example, the placement, position and/or configuration of the electrode device in relation to surrounding neurovascular tissues and/or organs, selection of energized electrodes on the device, and the pulse width, frequency and/or amplitude of the stimulation current, the user can identify an ideal stimulation scheme including an ideal implantation site with which the electrode device can affect in the kidneys, for providing desirable results in the regulation of blood pressure in the patient. In accordance with a feature of the present invention, the electrode device advantageously taps into an afferent pathway in the cervical region of the neck that leads to the brain for generating desirable efferent output in the abdominal region, including at least the kidneys. Desire efferent outputs include regulation of blood pressure and may include regulation of glucose and insulin production. Stimulation in the cervical region may further activate chemosensing activities in the cervical region, including, for example, a carotid body, which can be identified by changing levels of biomarkers and neurohormones, such as catecholamines, GLU, INS, GLP-1. The latter paves the way for diabetes therapy, including HTN and Insulin resistance/Type II diabetes, in addition to BP modulation for hypertension via the renal plexus, celiac plexus and/or splanchnic nerve.

### BRIEF DESCRIPTION OF THE DRAWINGS

The patent or application file contains at least one drawing executed in color. Copies of this patent or patent application publication with color drawings will be provided by the Office upon request and payment of the necessary fee.

These and other features and advantages of the present invention will be better understood by reference to the following detailed description when considered in conjunction with the accompanying drawings wherein:

FIG. 1 demonstrates known therapies relying on autonomic neuromodulation—stimulation of the parasympathetic nervous system or blocking the sympathetic nervous system.

FIG. 2 demonstrates an existing system and device that are adapted to perform renal sympathetic denervation.

FIG. 3 demonstrates another known system and device that rely on stimulation of the carotid sinus (baroreflex).

FIG. 4 is a graph summarizing results obtained with known systems, highlighting the efficacy of the therapy over a three year period.

FIGS. 5A-5E are photographs of an electrode device of the present invention in accordance with one embodiment, as implanted in an extra-vascular approach (deployment outside major vessels).

FIG. 6 is a perspective view of an electrode device of the present invention in accordance with one embodiment.

FIG. 7 is a perspective view of a single element assembly of the electrode device of FIG. 6.

FIGS. 8A and 8B are top plan views of a strap electrode device of the present invention in accordance with another embodiment.

FIGS. 9A-9F are photographs of a strap electrode device deployed in an abdomen, in a “tie and tuck” approach.

FIG. 10A illustrates placement of a self-deploying electrode device of the present invention in accordance with yet another embodiment.

FIG. 10B illustrates a small neck (circumference of about 28 cm) placement.

FIG. 10C illustrates a large neck (circumference of about 48 cm) placement.

FIG. 11A is a perspective view of an electrode device of the present invention in accordance with yet another embodiment, as hermetically sealed in a case.

FIG. 11B is a perspective view of the electrode device of FIG. 11A with the case shown in transparency.

FIGS. 11C and 11D are perspective views of the electrode device of FIG. 11A, with cuff members.

FIG. 11E are a series of cross-sectional views of the cuff members of FIGS. 11C and 11D.

FIG. 12 is a perspective view of an integrated electrode device of the present invention in accordance with yet another embodiment.

FIG. 12B is a view of the integrated electrode device of FIG. 12, as implanted in a patient.

FIG. 13 is a strap or patch.

FIGS. 14A and 14B are schematic illustrations of human anatomy, including the abdominal aorta and the celiac trunk, for an intravascular application of the present invention.

FIGS. 15A and 15B are schematic illustrations of human anatomy, including the aortic plexus, for an intravascular application of the present invention.

FIG. 16 illustrates a suitable implant site with percutaneous access from a patient's back.

FIG. 17. illustrates the aortic ganglion as a suitable implant site.

FIG. 18 illustrates the renal artery as a suitable implant site.

FIG. 19 illustrates a trans-oral approach used to access the abdominal area as a suitable implant site.

FIG. 20 is a block diagram of a system of the present invention in accordance with one embodiment.

FIG. 21 is a flow chart of a method of the present invention in accordance with one embodiment.

FIG. 22 is a dosing algorithm in accordance with the present invention.

FIG. 23 is a perspective view of a system for autonomic blood pressure regulation, in accordance with one embodiment of the present invention.

FIGS. 24A-24E are diagrams and charts of stimulation paradigms in accordance with an embodiment of the present invention, and their results.

FIG. 25 is an illustration depicting the cervical region of a patient.

FIG. 26 is a top plan view of a catheter adapted for use with the present invention, according to one embodiment.

FIG. 27 is an illustration of kidney neuromodulation via the Renin Angiotensin pathway, in accordance with one embodiment of the present invention.

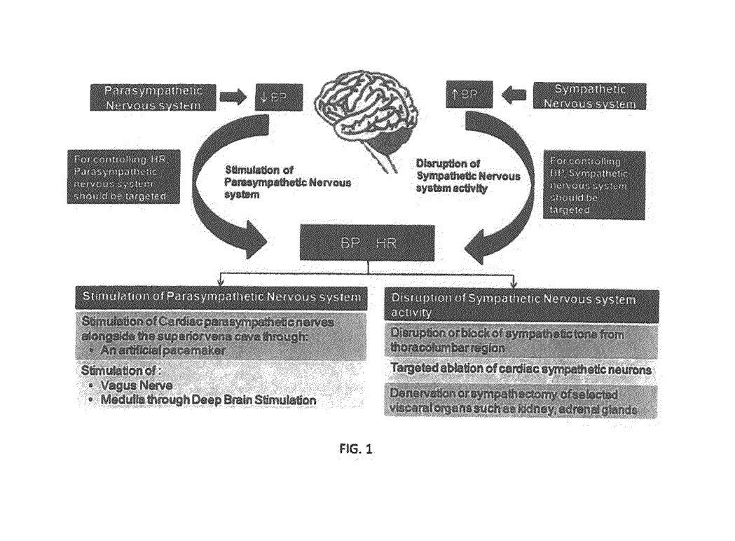

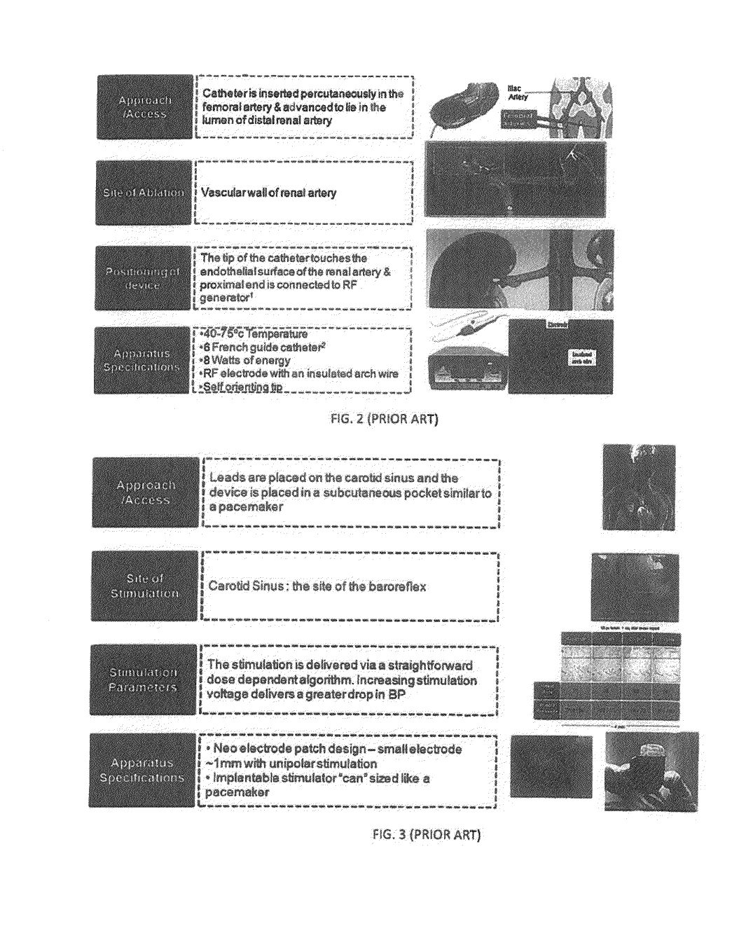

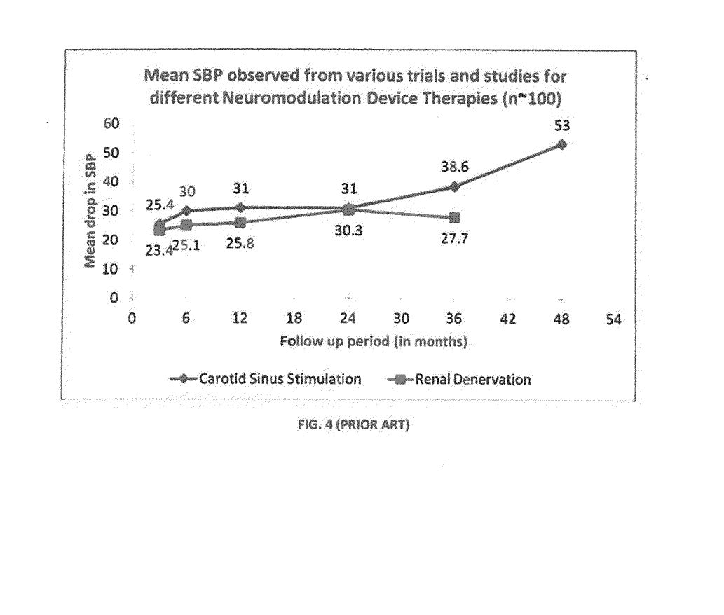

### DETAILED DESCRIPTION OF THE INVENTION

The present invention includes a “smart” electrode which could be self-deploying or have a lead-like configuration depending on the desired application which include intra-vascular or extra-vascular implant approach. The present invention includes a method applying downregulation of the sympathetic nervous system.

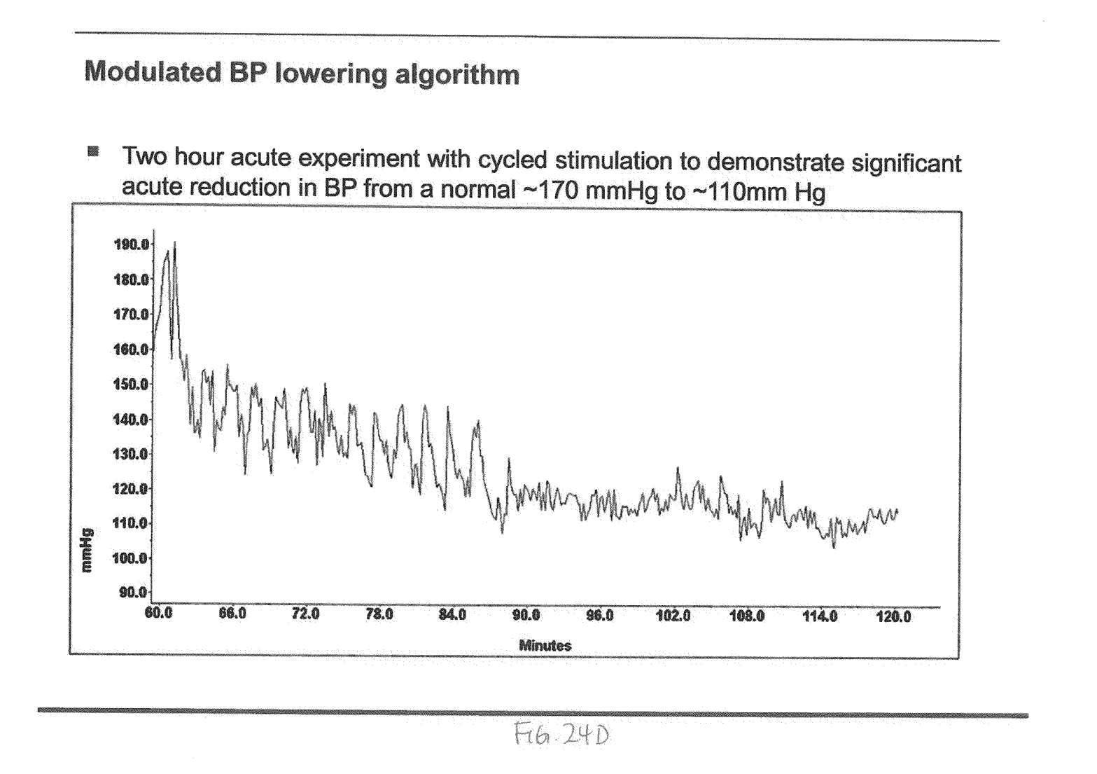

In one embodiment of the present invention as illustrated in FIG. 23, a system S includes an implantable electrode device 10, an implantable stimulation generator 15, a cable 29 with lead wires adapted to extend between and connect the device 10 and the generator 15. The system S may further include an implantable blood pressure (BP) sensor 21 (e.g., cuff) adapted for mounting on a vessel to determine blood pressure thereof. The system S may also include a remote transmitter 17 for remotely controlling the stimulator generator 15. The transmitter 17 may also be configured to power the generator 15 and/or recharge a battery (not shown) in the generator 15 and/or the BP sensor 21 via battery charger 27.

In alternate embodiments of the present invention, the device 10 and sensor 21 are engaged together with hybrid algorithms in order to keep mean average pressure (MAP) within a specific range. The device therapy may also be titrated to levels of circulating biomarkers like ANG II and rennin. For parasympathetic stimulation, frequency ranges to stimulate range from about 0-20 Hz, current from about 0-5 mA, about 0-0.5 ms, duty cycles ranging from about 10 s on/10 s off to about 60 s on/60 s off and doses may be delivered in time intervals ranging from about 0-24 hours. For neural blockade, this may be accomplished by specific arrangements of cathode-anode electrode arrays, or via high frequency nerve block (>about 100 Hz).

In one embodiment, a programmable, self-sizing electrode device is provided. The device may have individual programmable electrode elements that enable selective autonomic afferent/efferent fiber stimulation and recruitment.

Therapy may also be delivered via infusion pumps. The therapeutic within the pump may be one that stimulates or blocks sympathetic/parasympathetic nerves. For example, (NE, Epi, AChE, Serotonin) at night in order to essentially force MAP/SBP down at night and result in better control at night and during the early waking hours. The modulation of BP either via parasympathetic stimulation, sympathetic blockade or dual-use may occur via an active MAP sensor that may be placed anywhere in circulation but preferably in or near the heart or vessels with inner diameter >7 mm.

Steps to Enable the Therapy

Surgical Access

From an access standpoint, the renal sympathetic nerve may be accessed via the femoral artery or laparoscopically via the abdomen. The electrode device should have multiple active surfaces and a self-sizing design to enable the device to capture anatomic variation that may have more than one artery or ganglion bundle.

In one embodiment, the electrode device may be implanted sub-cutaneously in the abdomen. The electrode device may be placed on, adjacent or around a neurovascular structure, including a nerve, ganglia or a blood vessel. The device may be positioned or intravascularly.

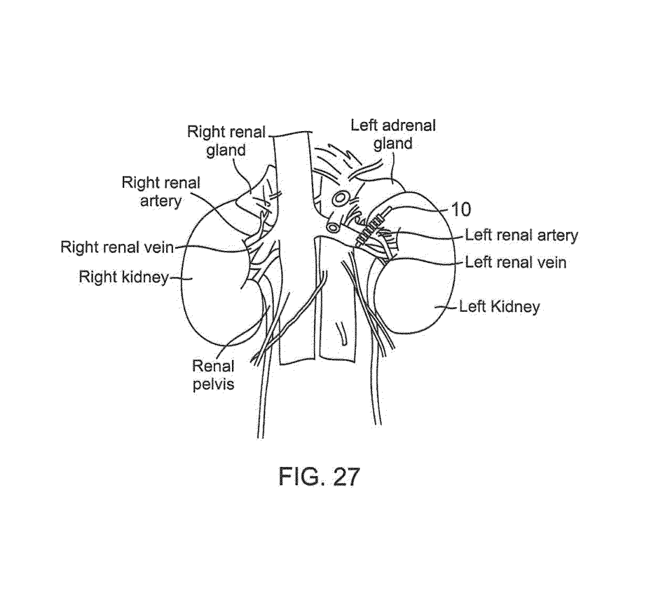

In one embodiment, as shown in FIG. 27, the electrode device 10 may be implanted subcutaneously in the abdomen, near the region of or in contact with the renal artery and/or renal vein for kidney modulation via the Renin Angiotensin pathway. The kidney and nervous system communicate via the renal plexus, whose fibers course along the renal arteries to reach each kidney. The electrode device 10 may be positioned to stimulate the renal plexus.

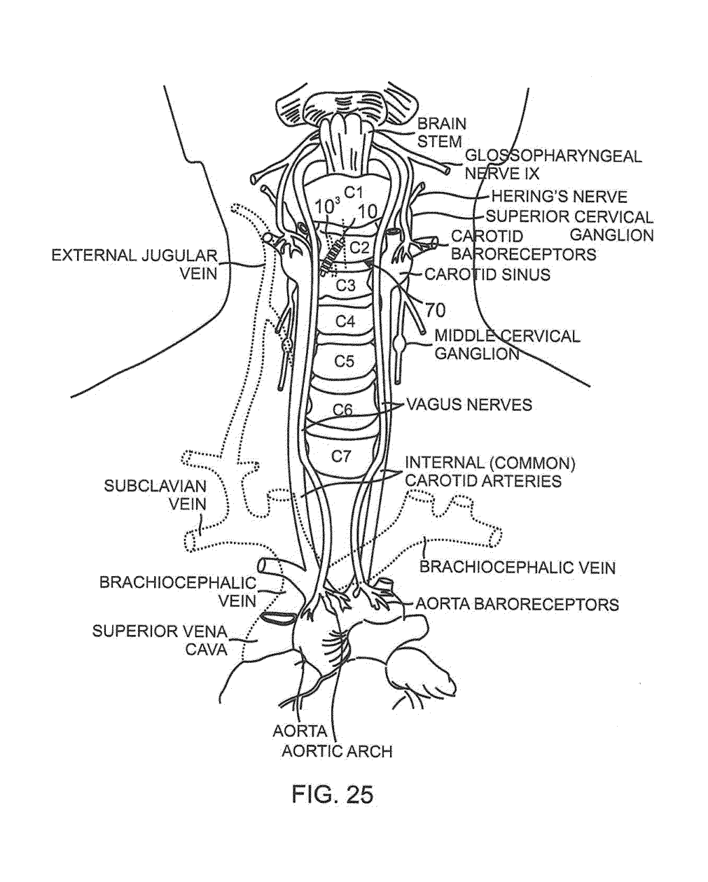

In another embodiment, the electrode device may be implanted subcutaneously in the cervical region 70, as shown in FIG. 25, preferably, near the cervical ganglia, between the common carotid arteries, above the aortic arch, and more preferably, in front of cervical vertebrae C2 and C3, near the carotid junction or carotid sinus. The device may be positioned so that any number of its electrodes near or come into contact with two or more tissue and/or organ structures, including plexi, such as the Hering's nerve, Vagus nerve, and carotid baroreceptors, and/or the brain stem.

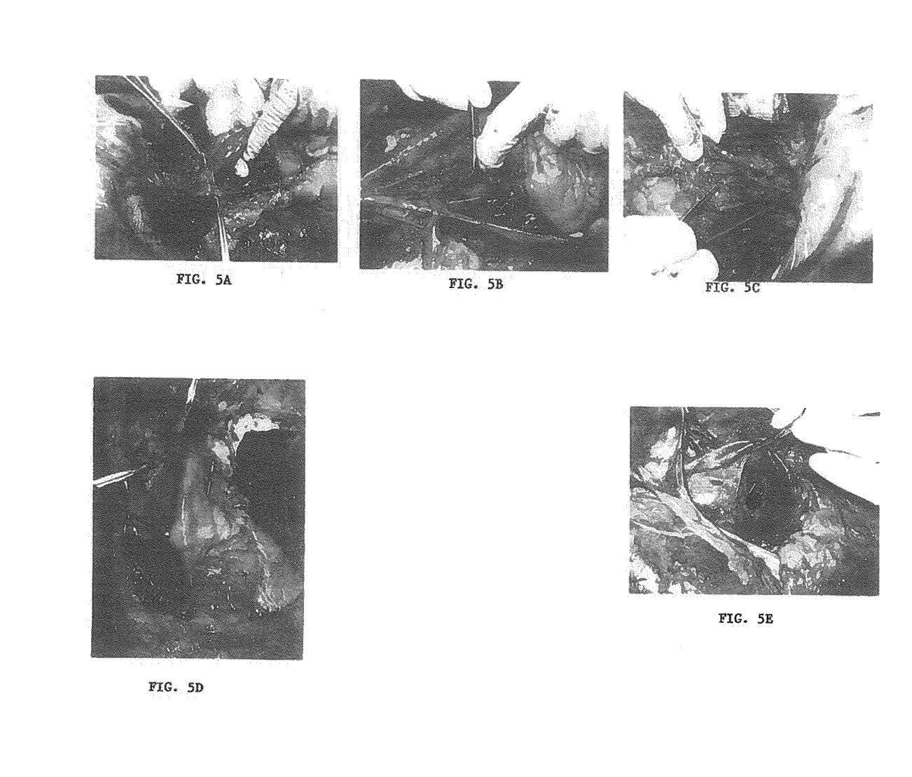

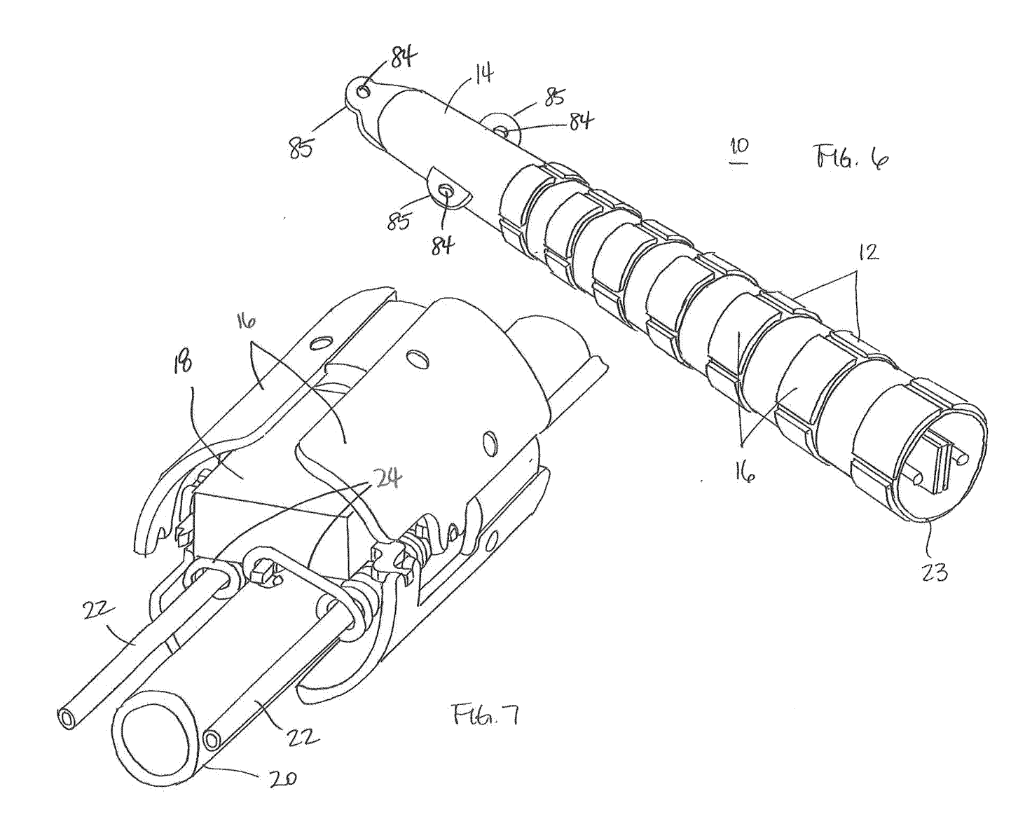

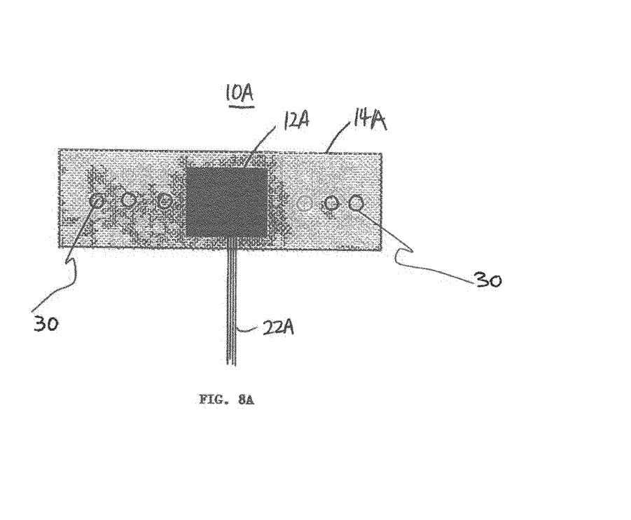

With reference to FIGS. 5A-5E, the electrode device 10 is deployed (on a delivery tool, e.g., guidewire or guiding sheath) around neurovascular structure, touching key plexus and large blood vessel (aorta). Power management may occur inductively or via ultrasound and the location is particularly important for this method to allow for good coupling and S/N ratio.

Also, in order to enable both sympathetic and parasympathetic control, the electrode device 10 may be designed to have “active” or transducing surfaces (or “electrodes”) to enable a charge density between 0.3-0.35 mC/cm and the arrangement of electrodes needs to be able to promote selective fiber recruitment. In one embodiment, a programmable electrode interface is provided to enable active selection of appropriate electrodes based on a closed-loop stimulus response functional test. As far as placement, the electrode device may be placed such that the active surfaces are in touch with BOTH sympathetic and parasympathetic nervous systems.

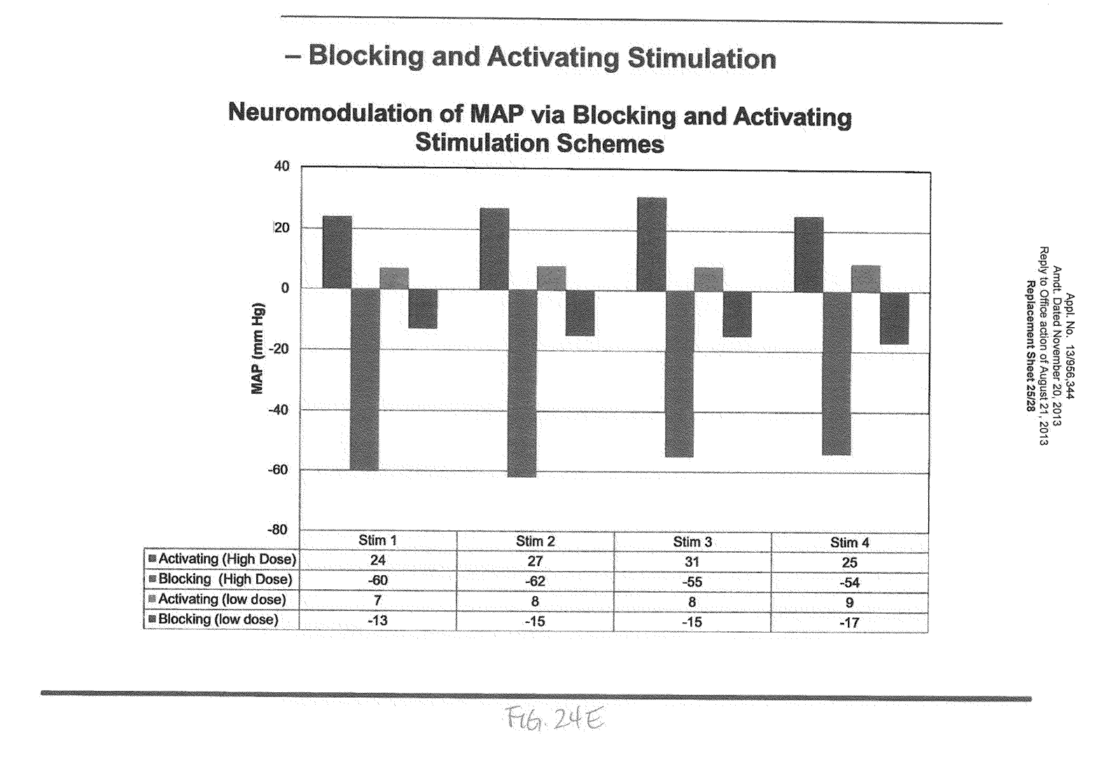

With reference to FIG. 6, the illustrated embodiment of the electrode device 10 employs a lead design with a plurality of “ring” electrodes or transducers 12 (for example, eight) mounted on a flexible elongated cylindrical body 14, having a nonconductive outer cover or tubing 23, with each transducer 12 having a plurality of modules 16 (for example, four covering about nearly all of 360 degrees radially) mounted on the tubing 23 in circumferential relationship with the body 14 such that the electrode device 10 has a plurality of contact surfaces at different positions longitudinally and different angles radially on the body 14. The electrodes/modules may be configured as monophasic or biphasic with selected pulse width(s), selected frequency/ies and amplitude(s) for providing selected duty cycle(s) generating results as shown in FIGS. 24A-24E. Lead body is about 5.4 Fr. Lead introducer is about 7 Fr. Distance between adjacent transducers 12 is about 10 mm.

With reference to FIG. 7, a single electrode element assembly is shown where each electrode module 16 may be constructed of Pt—Ir with TiNitride Coating. Housed in the tubing 23 (not shown) of the electrode device 10 is a hermetically sealed MEMS “chip in can” 18. Extending through the body 14 are a guidewire lumen 20 and cables 22 (e.g., MP35N DFT). Cables 22 extending longitudinally in the body 14 electrically connect each electrode 12 allowing the user to selectively activate one or more modules for stimulation. A coil 24 (e.g., of Pt—Ir) in surrounding relationship to one cable 22 is crimped to couple and secure the cables 22 and the MEMS chip in can 18 to each other.

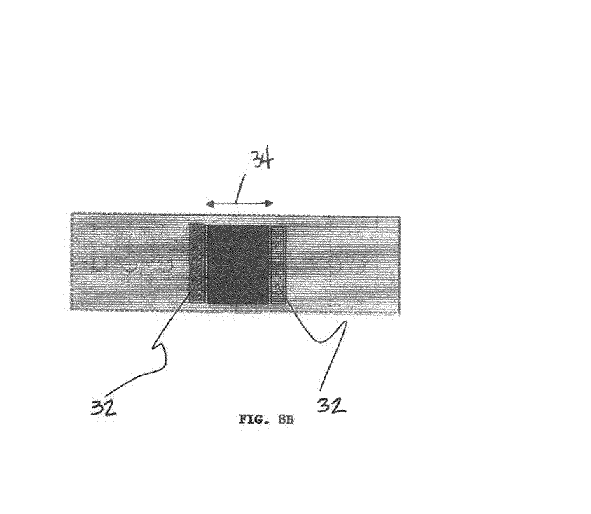

In an alternative embodiment, the electrode device may be adapted with flexible electrical circuits, active surfaces and power to be able to strap onto or around one or more neurovascular structures and be sutured into fascia (most likely abdominal fascia). With reference to FIGS. 8A and 8B, an electrode device 10A has a flexible rectangular strap body 14A with a lumen into which one or more electrodes 12A are inserted. The strap contains flexible electrodes along its length. Each electrode 12A preferably provides an effective surface area from about 1-5 cm squared in order to be able to activate tissue without any deleterious effects. Four cables 22A are twisted or braided for improved flexibility and no kinking. The cables have a strain relief that extends about six inches and ends in a terminal pin (not shown). A plurality of suture holes 30 may be provided in the strap body 14A for closing the strap body to make an internal diameter ranging from about 2-5 cm. Holes are punched along the strap at about 0.5 cm intervals to enable customization based on anatomy.

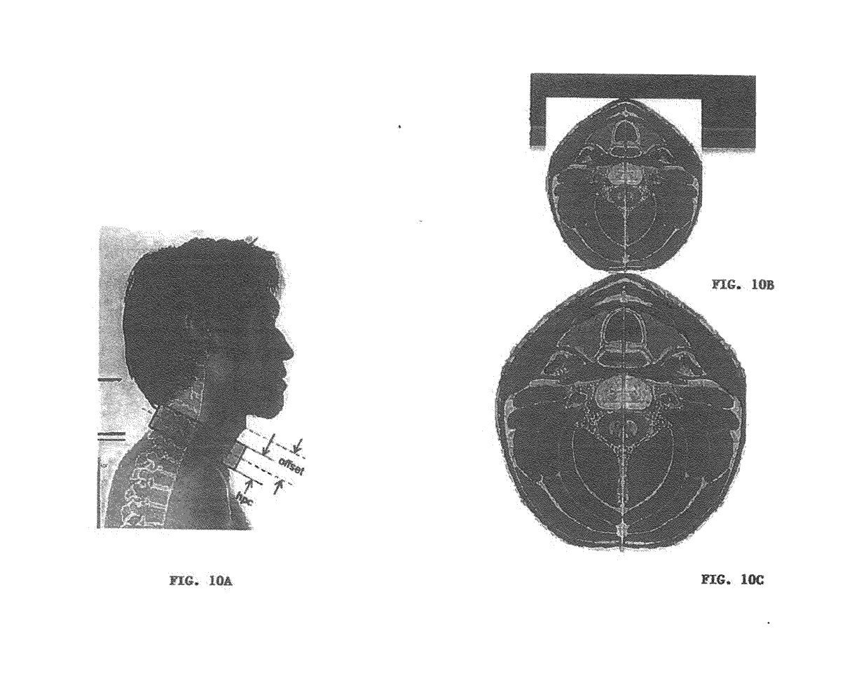

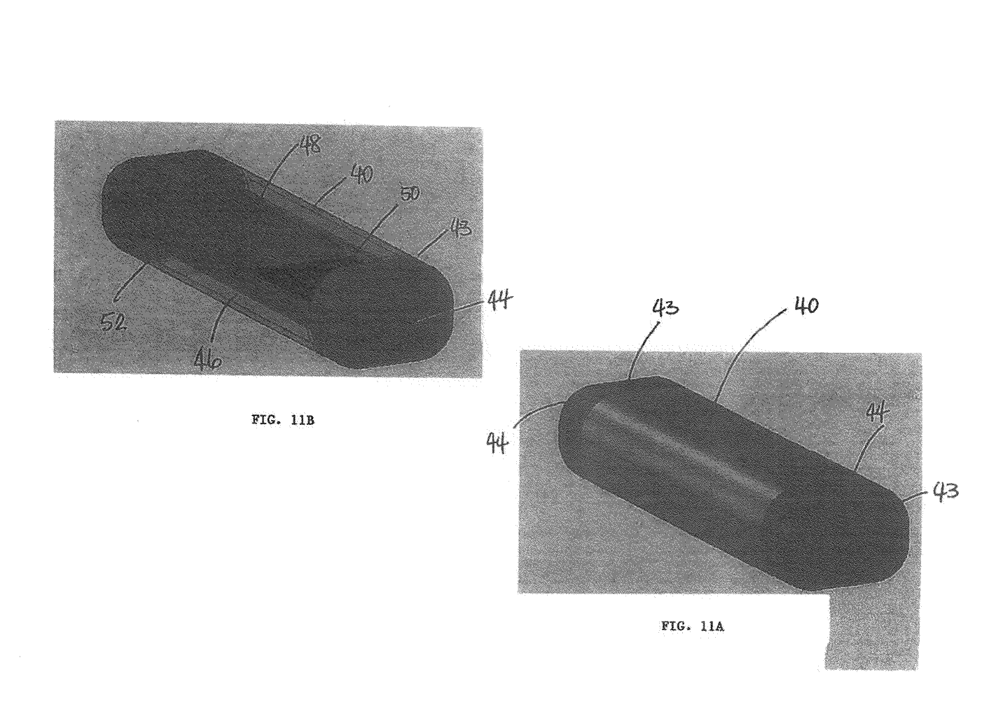

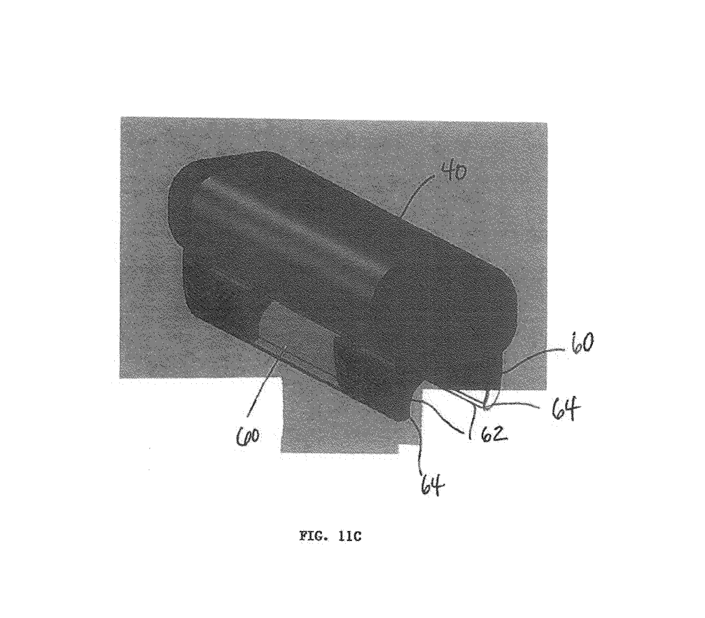

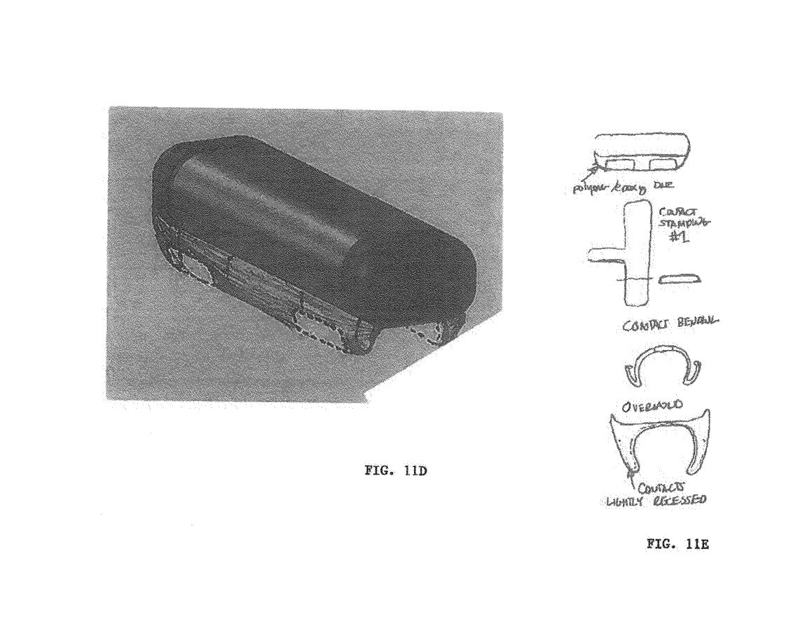

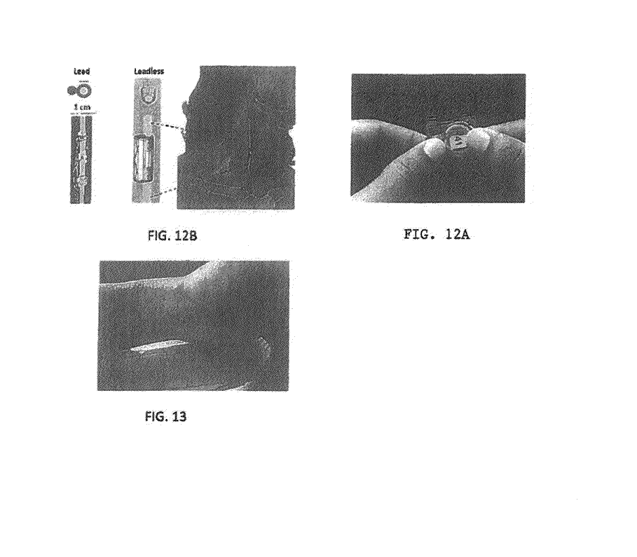

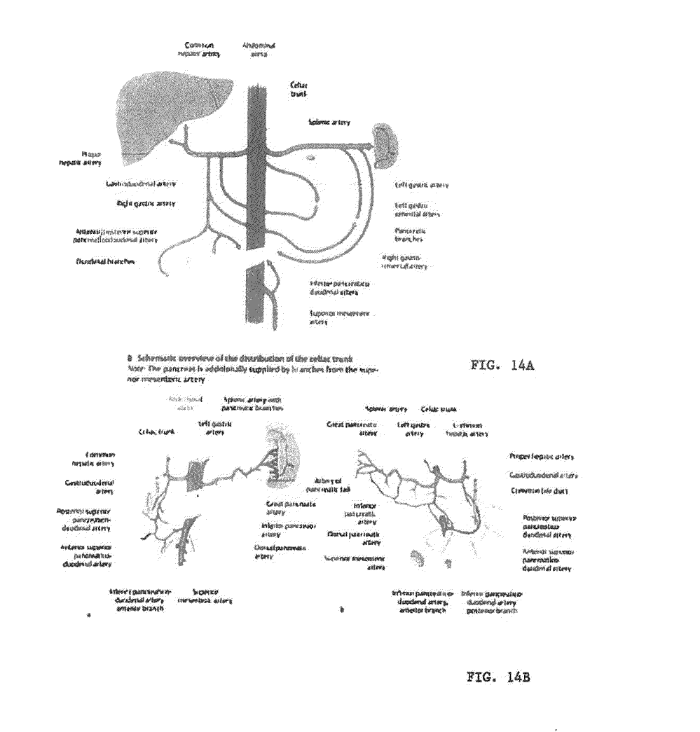

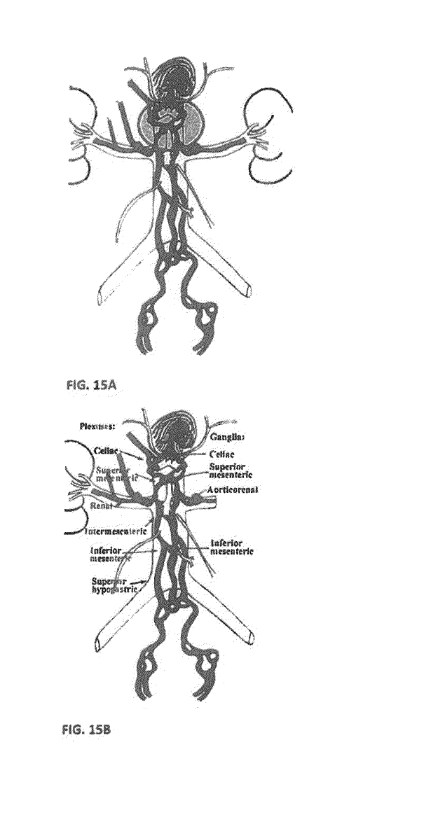

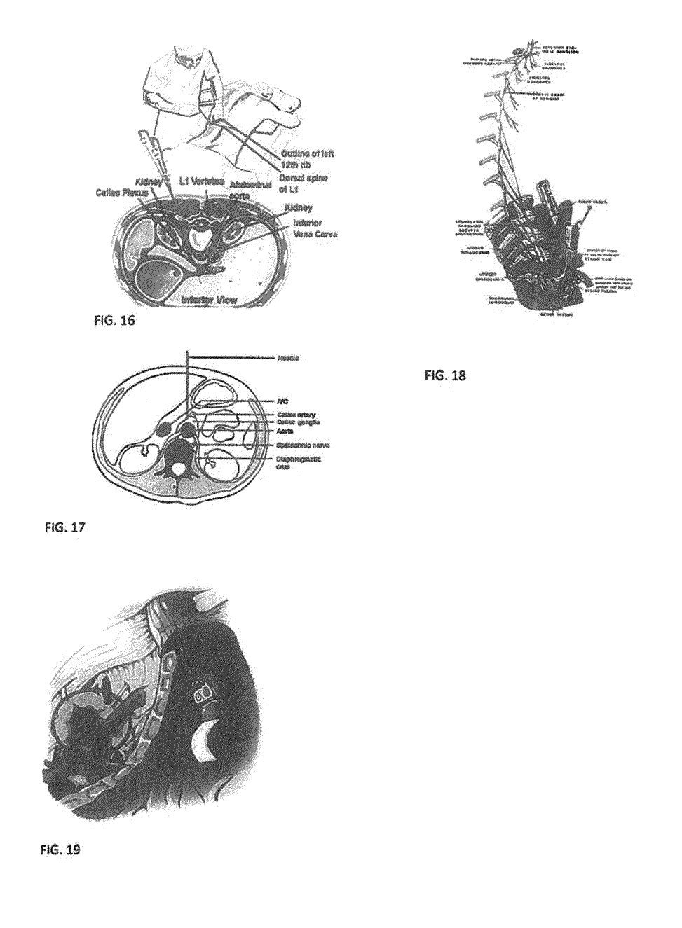

In another alternate embodiment, the electrode device may be self-deploying, as illustrated in FIGS. 10A, 10B, and 10C.

The electrode device 10B of FIGS. 11A-11B is sealed in a housing, for example, a ceramic case 40, having a tubular body 42 of a generally rectangular cross section. To hermetically seal the case 40, two end rings 43 and two end caps 44 of TiNb Braze are provided which can be laser welded to the tubular body. Housed therein is an electronic assembly 46 that slides in with spring contacts that is mechanically secure. The electronic assembly 46 includes a solenoid 48, a circuit board 50 (e.g., of hybrid thick film technology) and a battery 52 (e.g., solid state). A pair of modular opposing cuff members 60 with recessed contacts 62 and rounded leading edge 64 as illustrated in FIGS. 11C, 11D and 11E may be attachable to any surface of the housing by means of welds and/or glue, such as epoxy, to enable the device to “sit” or nest between or over neurovascular structures.

In yet another embodiment, an integrated electrode device 10C is illustrated in FIGS. 12A and 12B. Optional integrated contacts allow reimbursement to be the same as the aforementioned “lead” electrode device. This system also performs without an “active” POD. Flexible contacts 70 extend integral contacts around neurovascular structure such as a nerve.

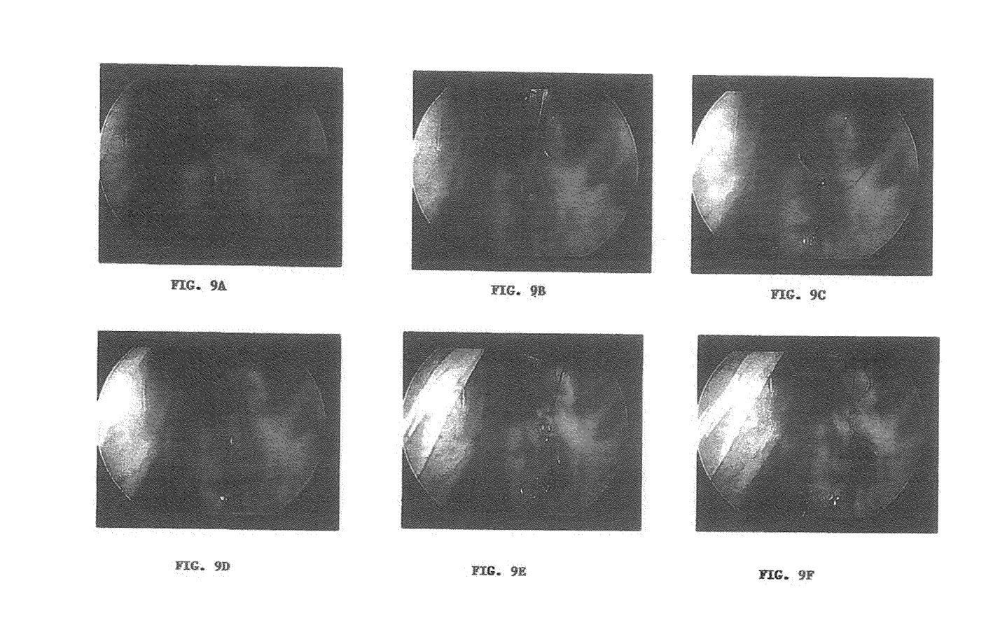

In addition, it is ideal that the blood pressure sensing (by the blood pressure sensor) be in the vicinity of the stimulator in order to promote close coupling. In the neck region, this includes being placed between selected neurovascular structure, including the vagus nerve, the carotid sinus and the stellate or cervical ganglia. With reference to FIGS. 9A-9F, in the abdominal region, an optimal location is near the aortic plexus, close to the suprarenal ganglia where the blood pressure sensing can occur from the large vessel and the electrodes of the device can be positioned on ganglia that have both parasympathetic afferents and sympathetic efferents. Recruitment profiles for these nerves may be differentiated based on permutations of constant current and frequency in order to allow for spatial and temporal firing patterns for neurons, as discussed in further detail below on Therapy Delivery.

The main aspects of surgical access include but are not limited to:

1. A location that has to allow the electrode device to have active surfaces in contact with both sympathetic and parasympathetic ganglia or direct paths to both. The location does not need to physically touch the specific ganglia because anodal or cathodal blocking schemes may be used to activate targets that are located up to about 2 cm away.

2. The electrode arrangement of the device involves about 4 or 8 active surfaces positioned on different surfaces of the electrode device similar to an array. Each of these electrodes may be programmed specifically to work synchronously or asynchronously with the others.

3. The blood pressure sensor may be co-located with the electrode device. It does need to maintain a minimum distance of 1 cm from the device in order to promote efficient wireless transmission within the space constraints. This is also why the system works in two specific locations in the body—the neck and the abdomen, immediately under the diaphragm but above the kidneys.

The electrode device may also be placed on the nerve via percutaneous access from the back or under fluoro, MR, CT guidance. There may be more than one electrode device that has to be placed on the branches of the renal artery (FIG. 18). FIG. 17 shows the location of the aortic ganglion that the electrode device may be placed on.

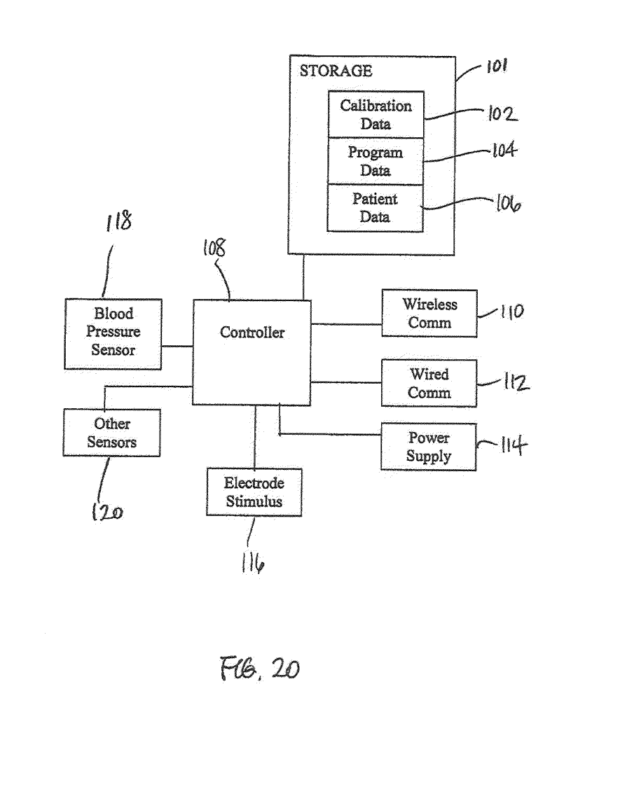

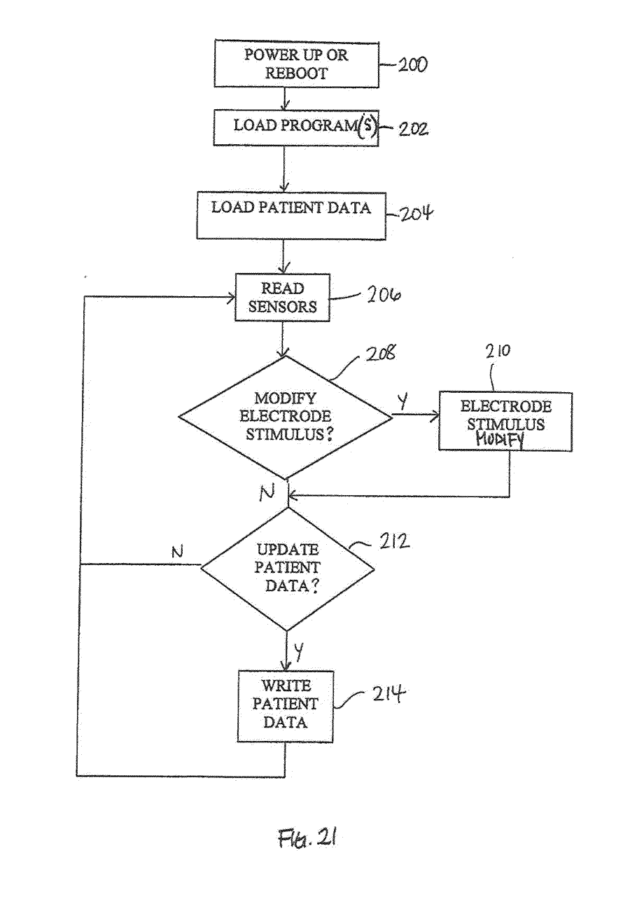

Alternatively, a trans-oral approach may be used to access the abdominal area where the electrode device may be placed. The access for this particular approach is shown in FIG. 19. The approach uses an endoscope and results in image-guided placement of the electrode device around the aorta ganglia below the heart and above the kidney as illustrated.

Implant Testing

Prior to placement of the electrode device, a functional test to confirm the location may be performed. This test involves a stimulus-response check and includes programmed protocols that the device executes automatically. These protocols typically involve monitoring changes in BP in a sense-and-measure paradigm. They are used to confirm device placement and optimal therapy programming as well as overall status and progress.

Some of the protocols include:

Delivery of an increasing amount of electrical stimulation (“ramping”) to the target, while monitoring muscle twitch to ensure patient comfort and determining the level at which a patient might feel activation or operation of the device. Specific frequencies to target about 0-4 Hz in order to be able to visualize the twitch as early as possible (simplest diagnostic). Alternative embodiments incorporate a stretchable electrode device on skin surface of the patient.

Delivery of an increasing amount of electrical stimulation to the target while monitoring the BP and HR to determine both safety and efficacy. The device is adapted for sense-and-deliver therapy and for selection from sympathetic-parasympathetic stimulation and blockade as well as efferent-afferent activation for determining the optimal response and self-selection based on that response. Each of the aforementioned permutations additionally involves various therapy parameters, including frequency, pulse width and duty cycle permutations. For instance, the optimal pattern for a parasympathetic firing may be a “burst stimulation” paradigm for about 10-20 s followed by about a minute off. During this minute off, a current ramped sympathetic blockade is applied with fibers recruited at about 3 mA, for about 0.5 ms, and current applied at >about 100 Hz.

Delivery of an increasing amount of stimulation to the target while monitoring analytes, for example, ANG II, rennin, GLU, INS, Alkaline Phosphatase. These analytes may be measured in real time with a MEMS chip or via a point-of-care system or via a MEMS chip implanted in a catheter. An example of some specific tests include a test to measure GLU and INS that involves delivery of a stimulus for about 5-10 minutes, followed by collections at about 0, 5, 10, 30 minutes in order to look at the gradient of the curve and confirm that gluconeogenesis or glycogenolysis are occurring over normal time courses and the autonomic nervous stimulation is not creating potentially unsafe situations. A standard OGTT may also be performed with the aforementioned stimulation protocol and this would be desirable if the therapy is extended to diabetes.

Customized patient profiles have an important role in the system. Each patient has a unique “setpoint” that is maintained by the autonomic nervous system. The electrode device is capable of moving a patient with chronic disease like hypertension to a more healthy and regular metabolic state via blocking of an inflammatory response and maintenance of appropriate cardiovascular and metabolic rhythm. In light of this “Metabolic Rhythm Management” customized sympathetic-parasympathetic tonic ratios may be maintained driven by BP, GLU, and bursts of INS and FFAs.

Therapy Modes

Delivery of stimulation to improve autonomic control of blood pressure and glucose is important to prevent exacerbation of a chronic condition. In chronic diseases like diabetes and hypertension, compensatory mechanisms intended for short-term control, result in long-term changes to the “metabolic” and/or “cardiovascular” set-point in the body. The autonomic nervous system plays a key role in many involuntary mechanisms that interact with neuro-hormonal messaging systems, cell membrane receptors, and the CNS in order to regulate a given “state” of the human body. It may be possible that symptoms for diseases like heart failure, diabetes, sleep apnea and hypertension can be greatly improved by manipulating the autonomic nervous system at key times of the day.

In one embodiment of the present invention, therapy involves delivering a sympathetic blockade during the night (defined by either sleep hours and/or darkness). Studies have shown the deleterious effects of increased BP during the night and high BP (and glucose) excursions in the morning. Some key elements of therapy delivery include but are not limited to:

Stimulation delivered to specific aortic ganglia located within 2 cm of the aorta, diaphragm, below the heart and above the kidneys. These ganglia may have some nerves connected to the T10-L1 region of the thoracic sympathetic chain and a percutaneous fluoroscopy-guided approach might also be possible. In this situation, a smart electrode designed like a “magic bullet” would be secured in the visceral fascia with a spinal approach from the T10-L1 area

Stimulation schemes that rely on an easy current ramp that will not cause pain or sensation to the patient. Pain and sensation can be prevented by selectively recruiting fibers, or by blocking pain afferents preferentially or by recruitment at low thresholds. An algorithm to select between these options in order to minimize power draw and receptor desensitization will select the best option. In one embodiment of this paradigm, a parasympathetic-stimulus may be deployed simultaneously with a sympathetic block. In another embodiment, a low-level tonic sympathetic stimulation may be applied as “background noise” to condition the system without raising MAP/HR by more than 5 mm Hg.

Stimulation schemes with specific doses that range from about 10-minutes to about 2 hours with firing patterns that mimic physiologic firing (bursts) mixed with intermittent tonic firing in order to prevent tachyphylaxis (sensitization of receptors)

Utilizing real-time biomarkers to set thresholds for stimulation. Practical considerations would involve selective recruitment of fibers that may bring down BP without affecting GLU levels, or raising BP without raising GLU levels. Recruitment profiles for various fiber types as well as temporal control via frequency is how this system improves overall control.

Therapy may be triggered by changes in blood pressure, heart rate, flow that may be sensed in a number of ways. One of the embodiments of the device senses changes in SNA, or in mechanical distension via strain/stress/displacement in a major blood vessel like the abdominal aorta or the jugular.

In one embodiment of the present invention, stimulation at a permutation of current, frequency and pulse width may be employed to preferentially activate a-delta fibers in order to reduce the ratio of sympathetic to parasympathetic tone. There are a number of tests that may be applied to test for this preferential activation. One such test is a stimulus-response test that is based on latency of a response (direct nerve recording). Another test involves an escalating ramp that stops the moment there is a change in cardiovascular status but below a threshold that will cause pain to the patient. The stimulation may be applied in “doses” that are dependent on a specific negative gradient and when the gradient=0 or close to zero (i.e. not much more of a fall in BP) the stimulation stops. In tandem with the stimulation is a heart rate sensor and a pain-threshold sensor. In the control unit, there will be triggers that are set to stop stimulation if C-fiber afferents (pain) are being activated or if there is any tachycardia or bradycardia.

In the most complex delivery of the therapy, sympathetic or parasympathetic stimulation and blockade of efferent or afferent impulses are delivered based on feedback from an implantable BP sensor. The general paradigm for therapy delivery involves dosing in increments that range from a few seconds to a few hours with a duty cycle. It is important that the therapy does not cause discomfort when it comes on and goes off—hence the need for a current ramp and selective frequencies to avoid sensation. Also, access plays an important role here because of the need for a plexus with sympathetic and parasympathetic connections—and the ability to read and transmit data from a sensor.

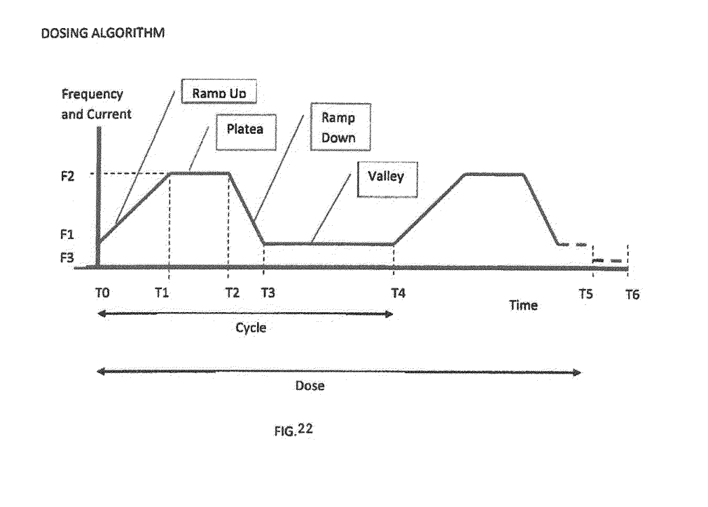

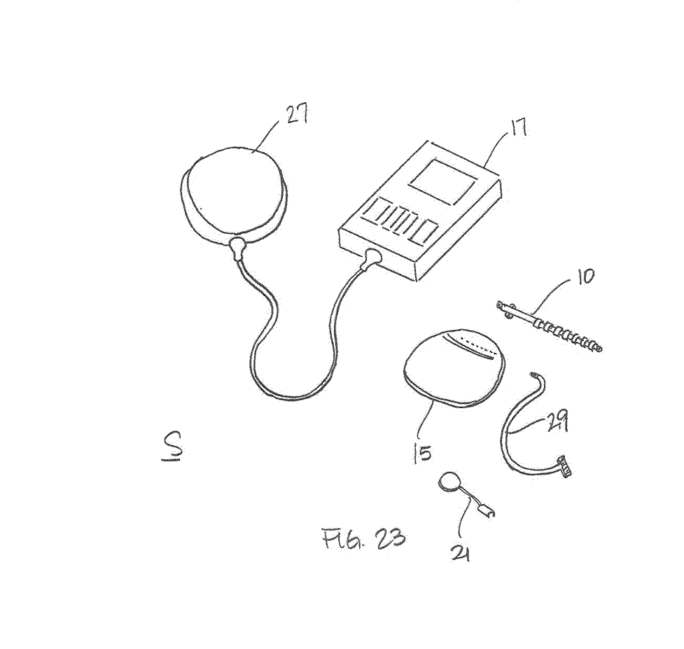

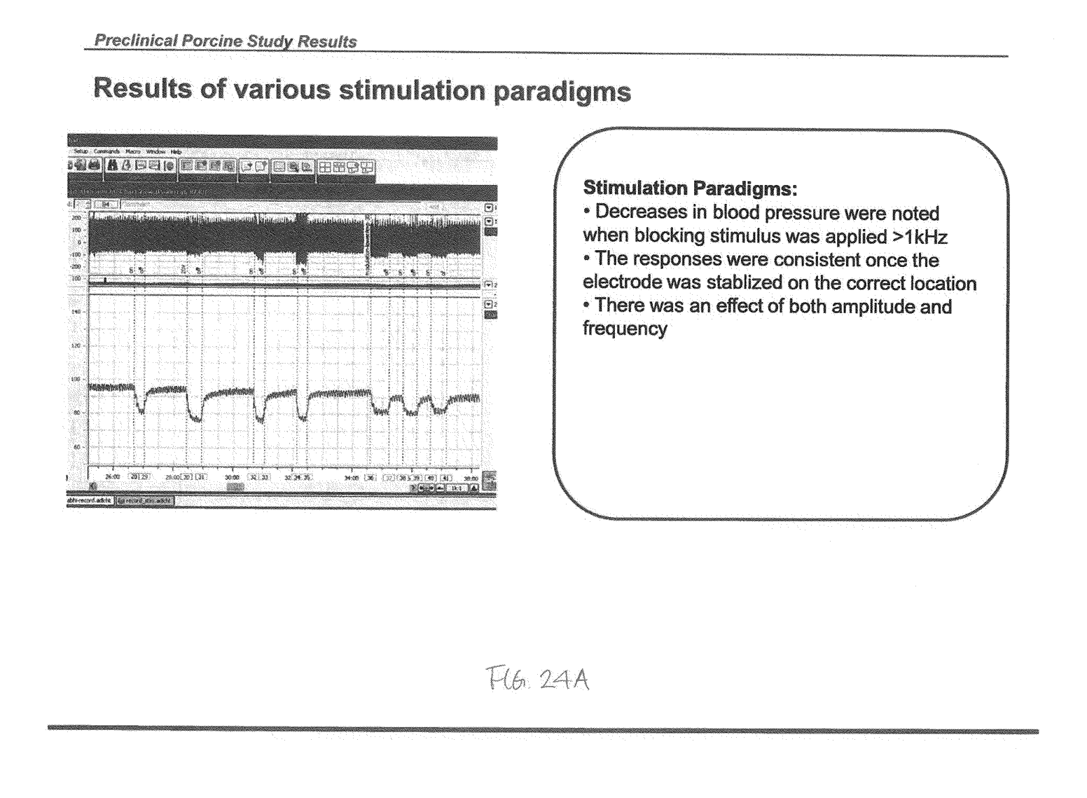

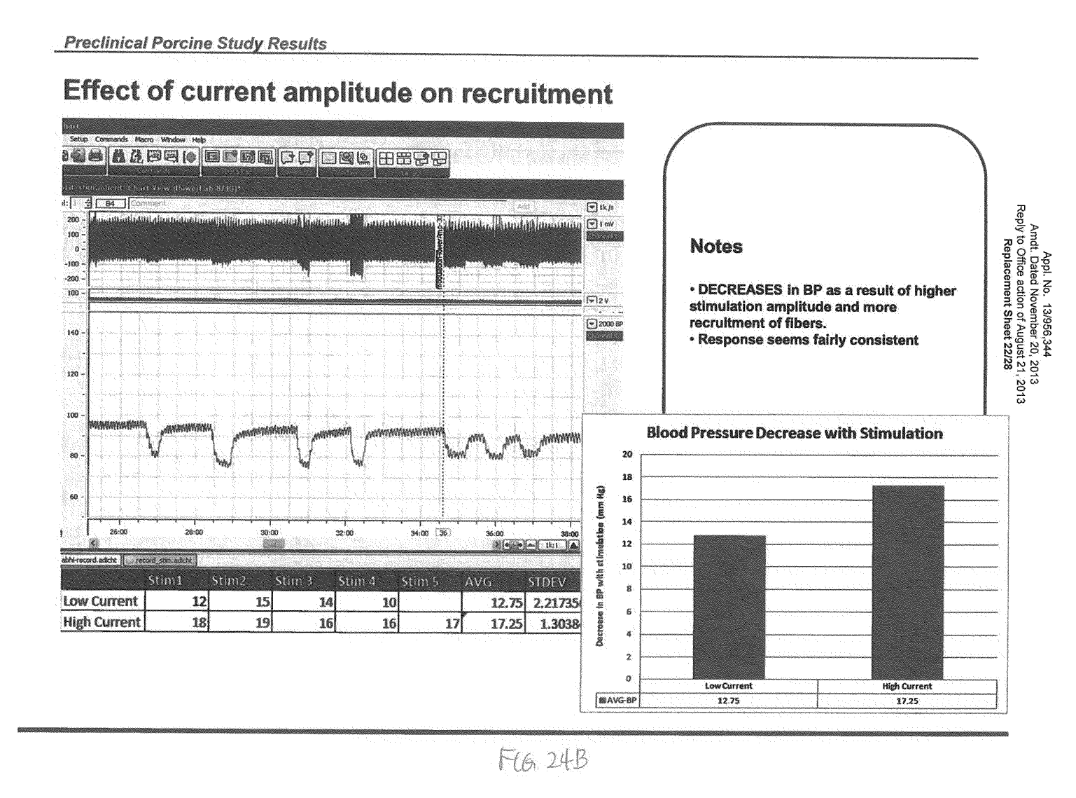

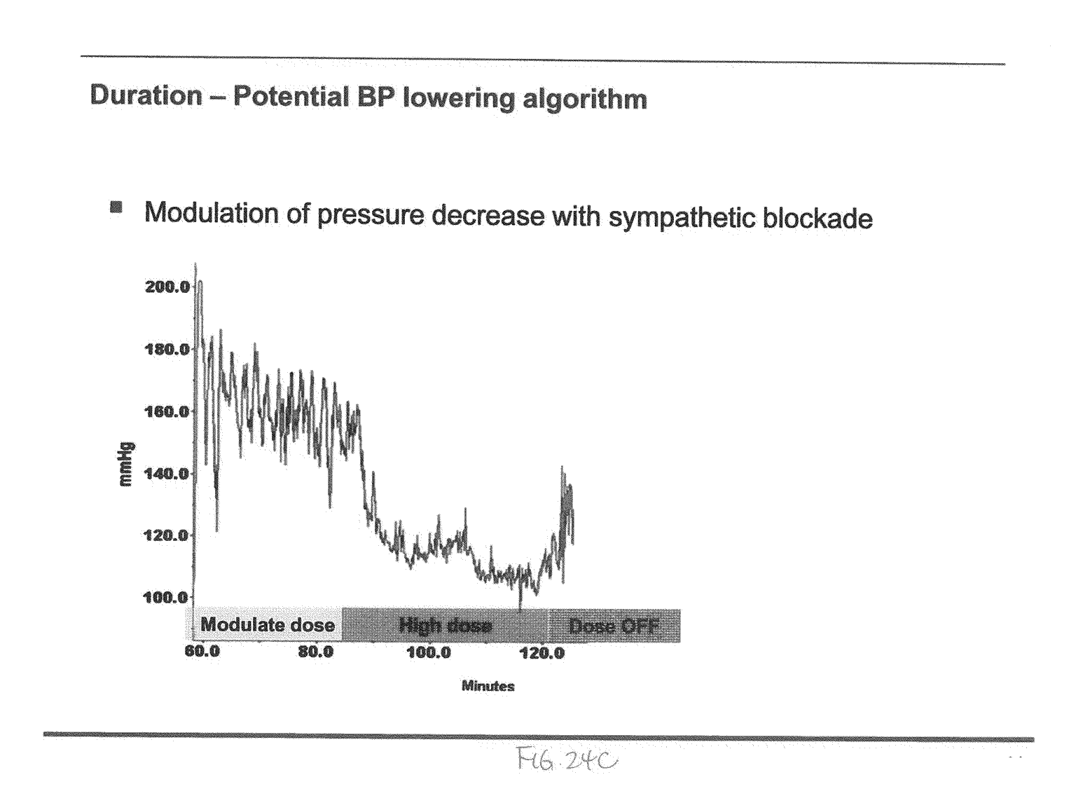

One dosing algorithm is illustrated in FIG. 22. During the daytime mode the circadian cycle deploys a repetitive stimulation pattern, consisting in a frequency ramp up, plateau, a ramp down and a valley and defined by the following programmable parameters: frequency, current and then doses that can be repeated over 24 hours.

Some key features of the present invention include:
Circadian Rhythm
Closed-loop control
Dose titration to MAP
Dose titration to Biomarkers (ANG II, rennin)

As for implant location, features and considerations include:
Sensor location
Device location
Minimally invasive access
Injectable percutaneous delivery
Nerve targets

The present invention advantageously features the following:
Target location—electrode stimulation between vessels in the neck and under the diaphragm
Applying stimulation to parasympathetic afferent nerve fibers to control sympathetic efferent nerve fibers. Specific permutation of frequency and current needed to accomplish this.
Functional mapping tied to fiber recruitment
Stimulus-response testing for BP, HR, rennin, ANG II, GFR and other biomarkers like GLP-1, TNF-alpha, cytokines, IL-1, IL-6
Ability to rotate through 90 and apply functional assessment to decide on appropriate placement of electrode
Sensor gated to therapy delivery
Therapy delivery without sensor

Night time dosing—Circadian algorithm.
Burst frequency
Ramping up to nominal current and down to zero
Electrode spacing
Electrode arrays for selective fiber activation

Test Paradigm

The present invention includes a functional mapping method in order to determine the appropriate level of stimulation (how much current, how much frequency), the appropriate target (sympathetic, parasympathetic, combination, efferent vs afferent and selective fiber activation) and the appropriate therapeutic regime/dosing strategy (high frequency nerve block vs. cathode-anode block)

In one embodiment, this method involves an electrode device with a multi-electrode array that can either rotate (enabling stimulation at different areas within, for example, a 3 cm×3 cm×3 cm cube) or has a multitude of active transducing elements that make rotation un-necessary.

In one embodiment, the electrode device is sandwiched between neurovascular structures, for example, the vagus nerve and the cervical plexus with the device being in contact with the vagus nerve on one surface and the plexus on the other. TAest paradigm may be employed in order to select the optimum electrode array stimulation. It is understood that the neurovascular structures may a parasympathetic plexus, a sympathetic plexus and a blood vessel with a diameter >5 mm that are within 5 cm of each other.

A number of thresholds may be calculated and customized for each patient and within a single patient, thresholds are calculated for both sympathetic and parasympathetic plexi. Some of the thresholds are—a muscle twitch threshold, a sensation threshold, a therapeutic threshold and optimum therapy threshold. A “threshold” may be a permutation of various parameters, including pulse width, frequency and current/voltage required to activate desired nerve fibers that correspond to that functional response (muscle twitch, BP, HR, rennin ANG II, GFR). A separate threshold may be calculated for sympathetic and parasympathetic because of the different effects of these targets and because the present invention utilizes automatic control as applied to mixed nerve targets.

A stimulus is delivered at a given set of parameters, including frequency, current (or voltage) and pulse width, and a biomarker is measured. The different biomarkers of interest have different time courses. For instance, assessments based on blood pressure or heart rate can be made in minutes and in some cases, seconds. Assessments based on GLU, INS, rennin and ANG II, and GLP-1 or immunomarkers may be made anywhere from about 5-min to 30-min. The frequency with which these markers are tested would likely be on a monthly timescale, while the blood pressure and heart rate (rapid-response biomarkers) may be checked and thresholds reset on a daily basis.

One of the challenges that may arise is that a given therapeutic threshold may be above a tolerance threshold for a patient. For instance, it could be that one needs a stimulation of 100 Hz (high frequency block) on a sympathetic plexi, at 3 mA, 0.5 ms to recruit the fibers required to lower the BP. However, this stimulation paradigm may be above the tolerance threshold for the patient. In this event, the therapy can be switched to a mode where the parasympathetic afferents may be engaged via a permutation of lower current (likely around 1-2 ma, 10 Hz) and maybe even a combination of cathode-anode-sympathetic block—to achieve the same effect (blockade of sympathetic efferent drive to lower blood pressure)

The other reason why this test paradigm and setting these thresholds automatically is desirable is because it directly impacts the anti-tachyphylactic algorithms that track changes in thresholds and improvements or worsening condition. For instance, over a 3-mo period, the threshold to achieve a given effect may move from 3 mA to 2 mA as a result of receptor downregulation and improvement in cardiovascular condition. Conversely, it may go for 3 mA to 4 mA to signal a worsening system—this is why tracking these thresholds over time is desirable for the autoadjust feature of the overall dosing algorithm

Ramping

Another feature of the present invention is increasing stimulation up to the appropriate dosing level. This is particularly applicable to the current and the pulse width since that controls the recruitment of nerve fibers. A sudden ramp (for instance, going from 0 ma to 3 mA in 1 s) is not desirable for blood pressure control because it would likely result in an increase or decrease in excess of 20 mm Hg. When one is trying to control BP, one has to “modulate” rather than abruptly increase or decrease. The ramp feature is designed to do the following:

a. Prevent sudden fluctuations in biomarkers. An example of a biomarker where a sudden fluctuation is detrimental to overall well-being is BP. A sudden increase in parasympathetic tone could cause cardiac rhythm disorders and it has been well-documented that fluctuations in BP is associated with increased risk of cerebrovascular disease. The ramp would prevent changes in BP of greater than 15% in 60 seconds.

b. Prevent stress and discomfort associated with onset of stimulation. Given the location of the electrode, there is ample opportunity for stray currents to stimulate a muscle twitch response in addition to stimulating pain or sensation afferent nerves. The ramp feature can last for up to about 1-hour in order to gradually work up to a specific stimulation threshold and gradually sensitize the nerves to the therapeutic threshold rather than engage the sensory threshold

Dosing Algorithm

The system and device apply a modular approach to neuromodulation—being able to combine features that correspond to physiology, e.g., the ramp up and down with the tonic underlying stimulation (baseline aka “Valley”—FIG. 22). Moreover, it may be desirable to have a “burst” of stimulation with tonic underlying blockade. The system and device also include integrating a Ramp-Up and Ramp-Down to prevent fluctuations in biomarkers and increasing or decreasing too quickly. The system and device may also include an exercise sensor to deactivate the system and/or electrode device during exercise. The system and device may also operate with a night-time mode to enable efferent blockade at night (defined as sleep hours and/or darkness), and be able to switch between stimulating parasympathetics and blocking sympathetic.

Anti-Tachyphylaxis

The system and device may feature autoadjust to set thresholds on a daily, weekly or monthly basis, based on cardiovascular system changes and measurement by the test paradigm. Example 1: As sensitization changes, it might be possible to stimulate for increased therapeutic benefit at higher levels of stimulation. Example 2: A person may have sympathetic plexi being “blocked” (100 Hz) at 3 mA. Maybe after 2 months of stimulation, it requires increased current to achieve the desired effect. The system may then opt to do one of three things: i) Supplement the existing sympathetic block with parasympathetic “burst firing” once every hour OR; ii) Increase the current in 0.1 mA steps (ramping) up to 0.5 mA or 1 mA (based on the test paradigm) OR; or iii) change the duty cycle or DOSE “OFF” time in order to provide the system with a ‘rest’ and downregulate receptors. During the DOSE OFF time, the system may deliver a burst of parasympathetic activity while the sympathetic side is being “rested.”

Multi-Electrode Stimulation

The system and device may be configured to provide stimulation with more than one smart electrode at more than one location. For instance, the system and device may be adapted to stimulate near the neck and under the diaphragm in order to change blood pressure in response to a pressure sensor. The system and device may also be adapted as renal perfusion is decreasing or as renal constriction increases to step-up increments of 0.1 mA stimulation (at a frequency >100 Hz) in the region near the celiac ganglia.

### Closed-Loop Stimulation

This paradigm is a variant where the test paradigm and the dosing algorithm are somewhat merged and control is established based on BP being in or out of range. Also, sensing may be accomplished by a blood pressure sensor that is part of this device, or belonging to another device (e.g., Left atrial pressure sensor or pressure sensor on a lead or integrated into an ICD or pacemaker). Moreover, the aim of the system and device is to maintain blood pressure within a specific tolerance range that may be set by a physician and potentially may include a “target set”. For instance, a person prone to HTN (Hypertension—high BP) where BP is around 160/110, may have a target set at 120/80 and in this case, the system and device is adapted to increase parasympathetic to sympathetic tone via a multitude of ways—by increasing current (0-5 mA) and frequency (100 Hz) to block sympathetic efferents or by increasing current and frequency (0-20 Hz) to boost parasympathetic tone. The “MAX” setting of the device to control a hypertensive patient is 5 mA, 100 Hz on the sympathetic nerve and 5 mA, 40 Hz on the parasympathetic plexus. At the latter, a burst stimulation paradigm may be used for maximal effect i.e. stimulation at 40 Hz maybe done for a few seconds followed by stimulation at 20 Hz for a few minutes. The same principles of the ramping and gradually working up to a stimulation threshold hold true here as well.

Furthermore, it is understood that the manner in which BP is brought back under control is a feature of the present invention. Fluctuations are minimized. Moreover, the system and device are adapted to regulate sympathetic and parasympathetic activity at the same time or at different times. For instance, to decrease BP, one can block sympathetic activity and do nothing to the parasympathetic side, or for maximum effect block sympathetic activity while increasing parasympathetic activity. Obviously in the latter stage, the dose cannot be delivered perpetually. A tonic sympathetic blockage may be engaged for chronic hypertensives but intermittent parasympathetic stimulation may be very beneficial. The functional test paradigm explained above is particularly useful for setting duty cycles and overall dose times. For instance, the parasympathetic stimulation may be engaged in a 30 s-on/30 s-off duty cycle for a period of 2-10 minutes every hour. This is an example of one “Algorithm” that is deployed for a chronic intractable hypertensive.

In the closed-loop scenario, when BP hits 140, the system and device access the sympathetic:parasympathetic ratio to determine whether it has changed. If it has increased, then a sympathetic block may be engaged or parasympathetic stimulation may be engaged—or both may be engaged depending on nerve activity (a biomarker), renal constriction, renal flow etc. A “default” scenario for high BP may be a gradual sympathetic block with increasing parasympathetic regulation if BP is not controlled within 2 hours. The ramping step-up increments to the therapeutic threshold making sure that SBP does not drop >10 mm Hg every minute. During this ramp control period, the BP sensor may read about every 5 secs and stimulation may be applied at a given current intensity and frequency for about 30 secs. The trend would be looked at together with the start and end for the 30 s “ON” and 30 s “OFF” and gradually stimulation may adjusted over each 30/30 “cycle” to get the BP closer to 120.

As illustrated in FIG. 20, a system 100 of the present invention in accordance with one embodiment includes a controller 108 in communication with a blood pressure sensor 118 and any other desired sensor(s) 120. The controller 108 is also in communication with an electrode stimulus 116 which may be embodied in the electrode device as described above. The controller is also in communication with a storage 101 which may include calibration data 102, program data 104 (such as the aforementioned methods), and patient data 106. The controller is may also be in communication with wireless communication device(s) 110, wired communication devices 112. The system 100 including the controller 108 may be powered by power supply 114.

As illustrated in FIG. 21, the system 100 may implement a method that includes power up as represented by block 200, followed by loading program(s) as represented by block 202, followed by loading patient data as represented by block 204, and followed by reading the sensor(s), including blood pressure sensor(s), as represented by block 206. The query is then posed of whether to modify the electrode stimulus (including the electrode device), as represented by query 208. If the answer is yes, then the electrode stimulus is modified as represented by block 210. If the answer is no, then the query of whether to update patient data is posed as represented by query 212. If yes, the patient data is written into storage, as represented by Block 214, and the system loops back to reading the sensors as represented by block 206. If the answer is no, then the system loops back to reading the sensors as represented by block 206.

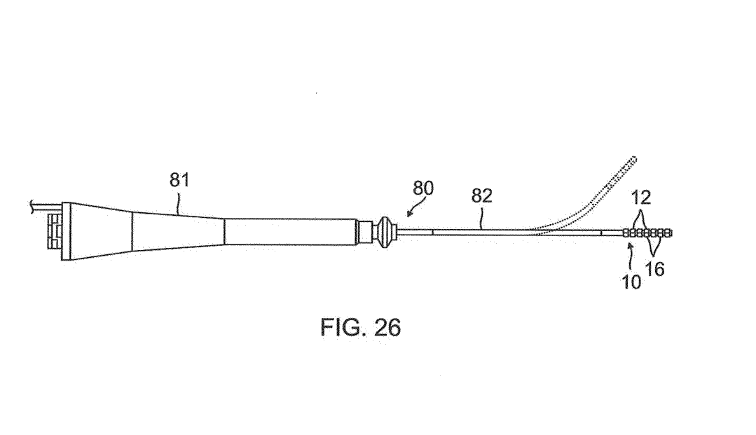

In use, the electrode device 10 may be releasably attached to a catheter 80 with steering capabilities, as shown in FIG. 26, for implantation in a patient's cervical region, as shown in FIG. 25, preferably, near the cervical ganglia, between the common carotid arteries, above the aortic arch, and more preferably, in front of cervical vertebrae C2 and C3, near the carotid junction or carotid sinus. An incision is made in the patient's neck region and the electrode device is advanced into the cervical region as a distal portion of the catheter 80 which is manipulated by puller wire(s) extending through the catheter that are responsive to a control handle 81 at the proximal end of the catheter, as known in the art. The catheter, and the electrode device 10 with multi-point/multi-channel capabilities, mounted on a distal end of the catheter shaft 82, are maneuvered by deflection of the catheter shaft 82 to a selected position (as shown at 10 in solid lines in FIG. 25) in the cervical region and real time, functional testing methods as described above are implemented to identify an optimal implantation site in relation to surrounding affected tissues and/or organs, including plexi, such as the Hering's nerve, Vagus nerve, and carotid baroreceptors, and/or the brain stem. That is, by selectively activating one or more electrodes 12/modules 16 to stimulate selected tissues and/or organs, and assessing in real time changes in activity/ies of the kidneys and/or blood pressure of the patient in response to the selected simulation, and then selectively activating one or more different electrodes 12/modules 16 and/or changing position of the electrode device 10 (as shown at 10′ in broken lines in FIG. 25) so as to contact different locations or different tissues and/or organs, and reassessing in real time changes in blood pressure, different stimulation schemes with different parameters are applied. Thus, through a methodical application of different stimulation schemes, including different permutations of, for example, the placement, position and/or configuration of the electrode device in relation to surrounding neurovascular tissues and/or organs, and the pulse width, frequency and/or amplitude of the stimulation current, the user can identify an ideal implantation site at which the electrode device can affect in the kidneys, for providing desirable results in the regulation of blood pressure in the patient. In accordance with a feature of the present invention, the electrode device advantageously taps into an afferent pathway in the cervical region of the neck that leads to the brain for generating desirable efferent output in the abdominal region, including at least the kidneys, if not also other organs in the abdominal area. Desire efferent outputs include regulation of blood pressure and may include regulation of glucose and insulin production. Stimulation in the cervical region may further activate chemosensing activities in the cervical region, including, for example, a carotid body, which can be identified by changing levels of biomarkers and neurohormones, such as catecholamines, GLU, INS, GLP-1. The latter paves the way for diabetes therapy, including HTN and Insulin resistance/Type II diabetes, in addition to BP modulation for hypertension via the renal plexus, celiac plexus, and/or splanchnic nerve.

With an ideal implantation site identified, the electrode device 10 may be detached from the catheter and attached to cable 29 providing connection to the stimulation generator 15 which may be implanted at a different location on the patient's body. The electrode device may be anchored by means of suturing to surrounding tissue, if desired, even though the electrode device may already be relatively lodged in place by surrounding bone and vasculature, such as, muscle, nerves and vessels, abound in the cervical region. Sutures may encircle the body 14 of the device 10 or the electrode device may have tabs 85 (see FIG. 6) formed on ends and/or sides of the body providing suture holes 84.

The BP sensor 21 (see FIG. 23) may be implanted in any suitable location in the patient's body for determining blood pressure and changes in response to the electrode device 10. With the electrode device implanted in the cervical region, a preferred implantation location for the BP sensor is on or near a major vessel in the cervical region, including, for example, a brachialcephalic artery, aortic arch, common carotid artery, or jugular vein. As known in the art, the BP sensor may be a flexible cuff that is mounted on a vessel and in communication and responsive to (preferably, wirelessly) to a controller housed in the stimulator generator 15

The preceding description has been presented with reference to certain exemplary embodiments of the invention. Workers skilled in the art and technology to which this invention pertains will appreciate that alterations and changes to the described structure may be practiced without meaningfully departing from the principal, spirit and scope of this invention. It is understood that the drawings are not necessarily to scale, and that features of the disclosed embodiments are interchangeable between different embodiments as needed or desired. Accordingly, the foregoing description should not be read as pertaining only to the precise structures described and illustrated in the accompanying drawings. Rather, it should be read as consistent with and as support for the following claims which are to have their fullest and fairest scope.

What is claimed is:

1. A method of assessing a site in a patient's body for implantation of an autonomic blood pressure regulating device, comprising:
selecting a neurovascular structure within the patient;
applying electrical stimulation with parameters to the selected neurovascular structure;
monitoring the selected neurovascular structure for at least one patient response to the electrical stimulation; and
adjusting at least one parameter of the electrical stimulation applied to the selected neurovascular structure in response to the patient response,
wherein the at least one patient response is selected from the group consisting of a muscle twitch, pain sensation, change in blood pressure, change in heart rate and change in an analyte.

2. The method of claim 1, wherein the neurovascular structure is selected from the group consisting of a sympathetic nerve, a parasympathetic nerve, the aortic ganglion, the vagus nerve, the carotid sinus, the stellate ganglia, the cervical ganglia, and the aortic plexus.

3. The method of claim 1, wherein the parameters include current, frequency, pulse width, duty cycle and off-duty cycle.

4. The method of claim 3, wherein the frequency ranges between about 0-4 Hz.

5. The method of claim 3, wherein the duty cycle ranges between about 0 to 5 seconds.

6. The method of claim 3, wherein the duty cycle ranges between about 1 to 5 hours.

7. The method of claim 3, wherein the duty cycle ranges between about 10 to 20 seconds and the off-duty cycle is about one minute.

8. The method of claim 3, wherein the current ranges between 3 mA and 5 mA.

9. The method of claim 1, wherein the analyte is selected from the group consisting of ANG II, rennin, GLU, INS, Alkaline Phosphatase, GLP-1, GFR, TNF-alpha, cytokines, IL-1, and IL-6.

10. An implantable electrode device for adjusting blood pressure, comprising:
a body member adapted for contact with one or more neurovascular structures and on a blood vessel; and
a plurality of transducers positioned on the body member, each adapted for selective application of electrical stimulation to the one or more neurovascular structures,
wherein the one or more neurovascular structures are selected from the group consisting of a sympathetic nerve, a parasympathetic nerve, the aortic ganglion, the vagus nerve, the carotid sinus, the stellate ganglia, the cervical ganglia, and the aortic plexus.

11. The device of claim 10, wherein the plurality of transducers ranges between about 4 to 8.

12. The device of claim 10, wherein the one or more neurovascular structures includes at least one sympathetic nerve and one parasympathetic nerve.

13. The device of claim 10, wherein the device is positioned intravascularly between the one or more neurovascular structures.

14. The device of claim 10, wherein the device is mounted on one or more neurovascular structures.

15. The device of claim 10, further comprising a blood pressure sensor mounted on the blood vessel.

16. A method of treating hypertension in a patient, comprising
(a) implanting the device of claim 10 in the patient;
(b) measuring blood pressure of patient;
(c) selecting one or more transducers;
(d) applying electrical stimulation to respective neurovascular structures via the selected one or more transducers, the electrical stimulation defined by parameters including current, frequency, pulse width, duty on cycle and duty off cycle;
(e) adjusting one or more of the parameters in response to the measured blood pressure; and
(f) repeating (b) through (e).

17. The method of claim 16, wherein selecting one or more transducers includes selecting a transducer for applying electrical stimulation to a sympathetic nerve while the patient is asleep.

18. The method of claim 16, wherein selecting one or more transducers includes selecting a transducer for applying electrical stimulation to a parasympathetic nerve

19. The method of claim 16, wherein applying electrical stimulation includes adjusting the current in accordance with a ramp algorithm.

20. The method of claim 19, wherein the electrical stimulation is applied in a predetermined manner mimicking physiologic firing.

21. The method of claim 20, wherein the predetermined manner includes intermittent tonic firing.

22. An implantable electrode device for regulating blood pressure, comprising:
a body member adapted for implantation in a cervical region of a patient; and
a plurality of electrodes positioned on the body member, each adapted for selective application of electrical stimulation to the cervical region;
wherein the cervical region is defined generally between a pair of common carotid ateries, above an aortic arch and in front of cervical vertebrae C2 and C3

23. A method of implanting a device adapted to treat hypertension in a patient, comprising:
(a) placing the device of claim 22 in the cervical region;
(b) energizing the device in accordance with a stimulation scheme defined by a plurality of parameters;
(c) determining any change in blood pressure of patient;
(d) varying at least one parameter to define a different stimulation scheme.
(e) repeating (b) and (c).

24. The method of claim 23, wherein the plurality of parameters are selected from the group consisting of: position of device in the cervical region and selection of at least one or more electrodes on the device being energized.

25. The method of claim 24, wherein the group further consists of: neurovascular structures in contact with the selected electrodes, frequency of stimulation energy, pulse width of stimulation energy, duty on cycle of stimulation energy and duty off cycle of stimulation energy.
